(19) United States
(12) Patent Application Publication
(10) Pub. No.: US 2009/0205107 A1
(43) Pub. Date: Aug. 20, 2009

(54) Cooling Headgear

(76) Inventor: Nelson Coba, Pembroke Pines, FL (US)

Correspondence Address: ROBERT M. SCHWARTZ, P.A., P.O. BOX 221470, HOLLYWOOD, FL, 33022, US

(21) Appl. No.: 12/364,618

(22) Filed: Feb. 3, 2009

Related U.S. Application Data
(60) Provisional application No. 61/029,616, filed on Feb. 19, 2008.

(51) Int. Cl. A42C 5/00; A42B 1/24; A61F 7/00; A42B 1/06

(52) U.S. Cl. 2/171.2; 2/209.13; 607/109; 607/112; 2/172

(57) ABSTRACT

The present invention is an hat for cooling the head of a wearer having a chamber comprising a central cavity and at least one opening for placing ice into the cavity; and at least one porous member secured to one end of said chamber; wherein the apparatus is configured to be demountably attached to the head of a wearer.

### INDEX TO RELATED APPLICATIONS

This application claims benefit of U.S. Provisional Patent Application No. 61/029,616 filed Feb. 19, 2008, the disclosure of which is incorporated herein by reference in its entirety.

### BRIEF SUMMARY OF THE INVENTION

According to the present invention there is provided a headwear article useful for cooling its wearer which comprises a head fitment portion and at least one pouch portion. The pouch portion includes a compartment disposed on the crown portion of the head of the wearer. The compartment is configured to contain ice or other suitable cooling material. As ice in the compartment melts due to exposure to ambient air, the water from the melted ice diffuses through the compartment and further diffuses into at least one dispersement member. The cool water diffusing through the disbursing member positioned about the head and neck of a wearer creates a cooling effect and provides comfort on hot days, during work, or exercise activities. The head fitment portion of the invention may include any article of headwear known in the art.

In a preferred embodiment, the invention is a hat for cooling the head of a wearer having at least one chamber with a central cavity having at least one opening for placing ice into said cavity and at least one porous member secured to one end of said chamber; wherein the chamber is configured to be demountably attached to the head of a wearer.

In a preferred embodiment, the chamber is positioned adjacent to the crown of a cap. The chamber includes a mechanism for closing and securing ice on the interior of the chamber. The mechanism may be a zipper, buttons, snaps, hook and loop fastener such as Velcro® and the like. Preferably, the chamber is at least partially porous to allow moisture from the interior of the chamber to diffuse into the porous dispersion member.

The diffusion of moisture occurs at least in part at the interface where the porous dispersion member is attached to the chamber.

The dispersion member may be made of cloth, terrycloth or any suitable material having porosity to permit diffusion of water therethrough.

### BRIEF DESCRIPTION OF THE DRAWINGS

FIG. 1 is a front view of the present invention worn by a person.

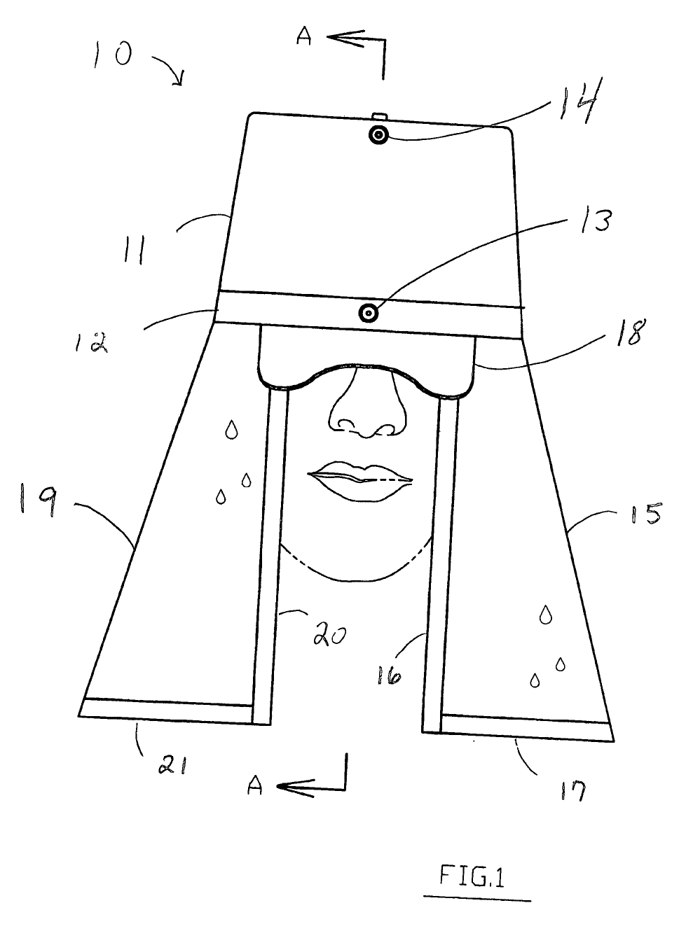

FIG. 2 is a top view of the present invention.

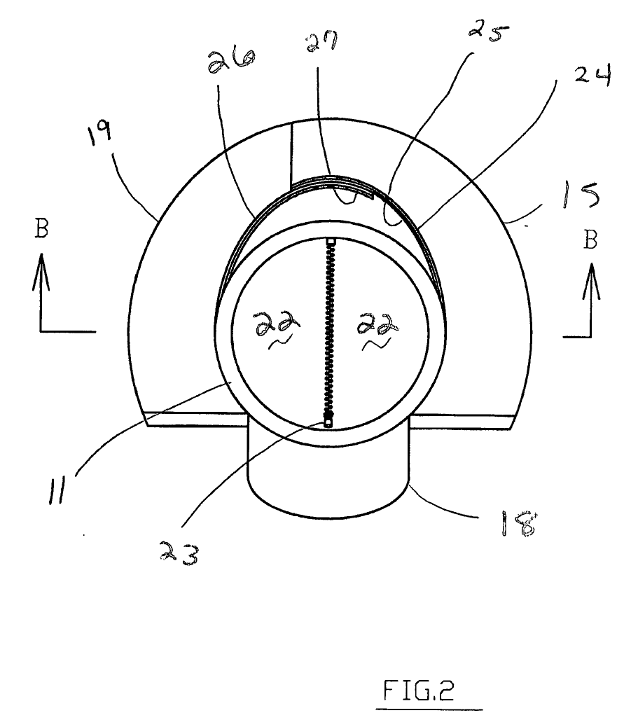

FIG. 3 is a side view in partial cut away along section line A-A from FIG. 1.

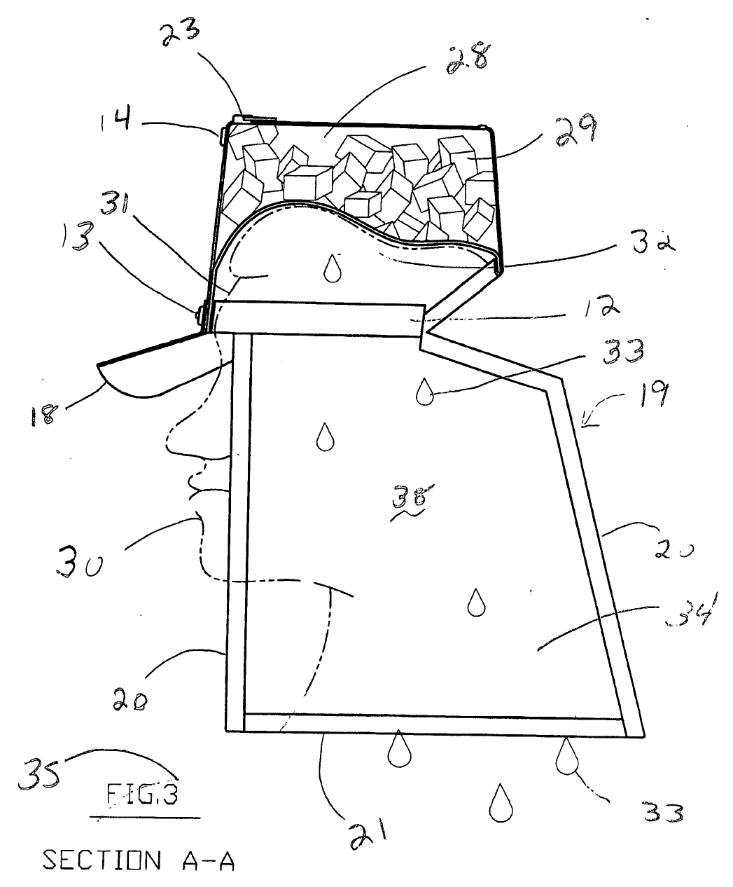

FIG. 4 FIG. 3 is a front view in partial cut away along section line B-B from FIG. 2.

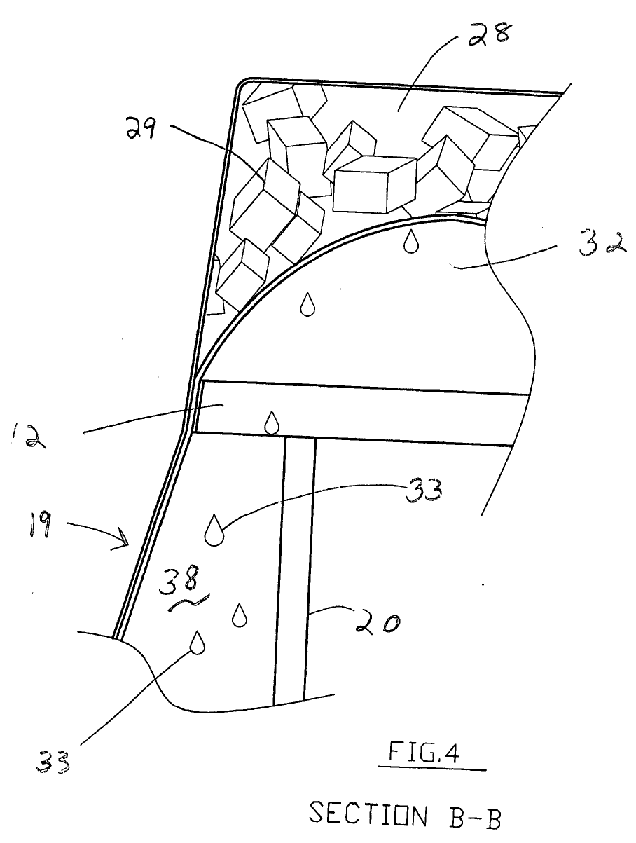

FIG. 5 is a side view in partial cut away showing the ice storage cavity.

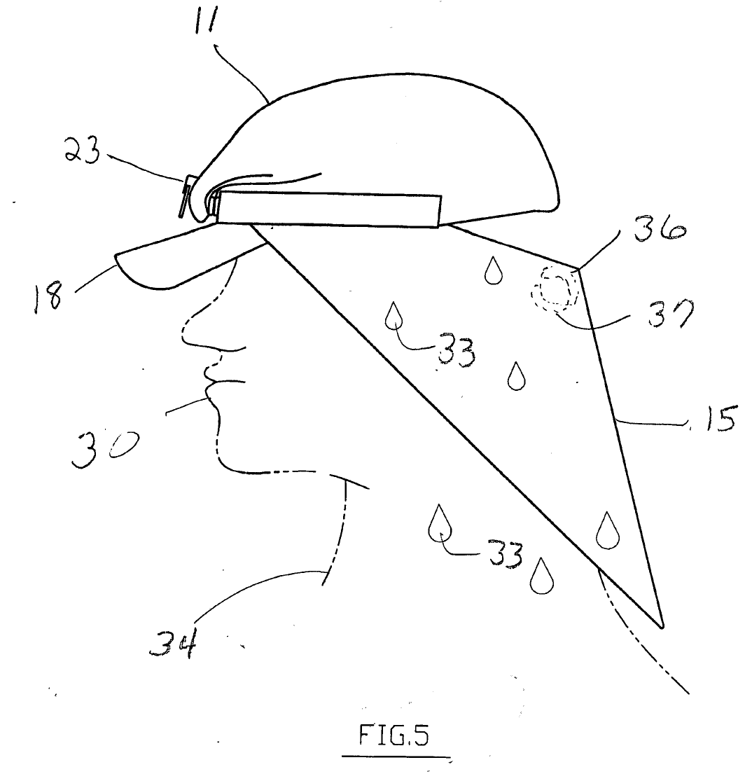

FIG. 6 is a side view showing the shade pulled back and attached to itself.

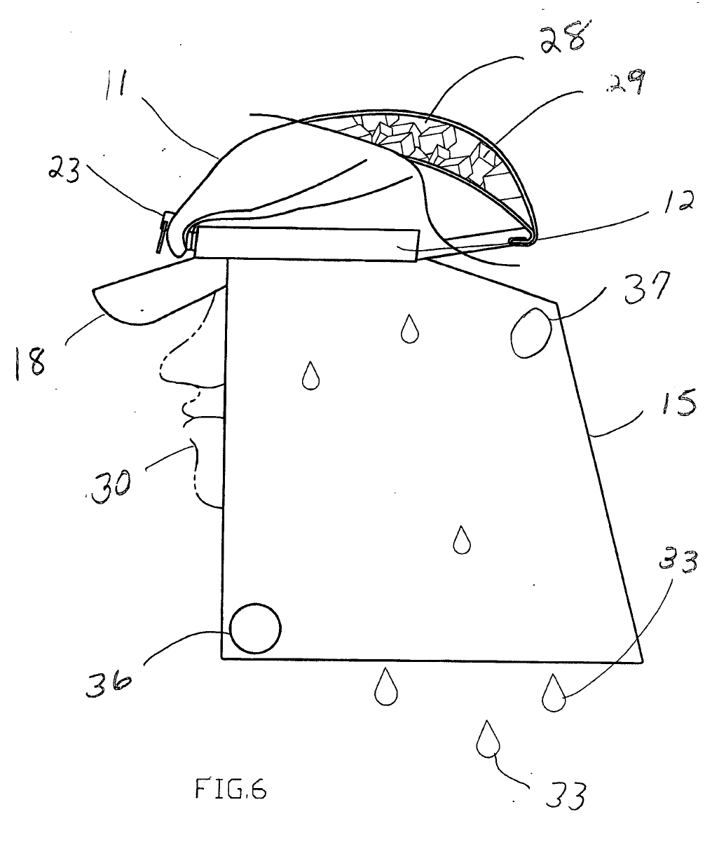

### DETAILED DESCRIPTION OF THE PREFERRED EMBODIMENTS

Hat 10, has a crown 11, that is circumferraly bound by band 12 on the lower edge of crown 11. Band 12, has a male snap 13, and a corresponding female snap 14, is affixed to crown 11. Hat 10 includes brim 18. Hat 10 has a first shade 15 that is affixed on its upper edge to crown 11 at band 12. First shade 15 has vertical trim 16 and lower trim 17 attached about the perimeter of first shade 15. First shade 15 has a portion along its upper edge with upper trim 24 that is not affixed to band 12. Upper trim 24 has, at least a portion thereof, having a loop faster 25 that affixes to a corresponding hook 27 fastener on the upper trim 26 of second shade 19.

Hat 10 has a second shade 19 that is affixed on its upper edge to crown 11 at band 12 has a portion along its upper edge not affixed to band 12. Second shade 19 has vertical trim 20 and lower trim 21 attached about the perimeter of second shade 19.

Hat 10 has crown cavity 28 encompassing at least a portion of the interior region of crown 11. Crown cavity 28 is constructed and arranged to accommodate ice 29 placed therein.

Band 12 surrounds the circumference of forehead 31 and the head 32 of a user 30.

A zipper 23 is slidably moved to allow access to crown cavity 28. Crown cavity 28 is filled with ice 29 and zipper 23 is closed to prevent ice from exiting cavity 28 an to insulate the interior region from warmer atmospheric conditions on the outside of hat 10. As ice 29 melts, water droplets 33 form. Formed droplets 33 fall to the lower portion of cavity 28. Each of first shade 15 and second shade 19 are attached to crown 11 at band 12. Each of first shade 15 and second shade 19 are constructed and arranged with an interior porous liner 38 that faces inward toward a wearer 30 and adjacent to face 30 and neck 34 of the wearer. Interior porous liner 38 has an upper terminal end in crown cavity 28 that extends beyond band 12. In one embodiment, the interior portion of crown 12, that contacts head 54, also has, at least some portion of interior porous liner 38 affixed thereto.

Water droplets 33 from melting ice 29 transpire into interior porous liner 38 and gravitationally travel downward inside porous liner 38 with a wicking effect that moisturizes substantial portions of interior porous liner 38.

A user 30 wearing hat 10 in which substantial portions of interior porous liner 38 are wet will experience a cooling effect around the head, face, and neck.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An hat for cooling the head of a wearer comprising:
a. a chamber with a central cavity having at least one opening for placing ice into said cavity;
b. at least one porous member secured to one end of said chamber;
wherein said chamber is configured to be demountably attached to the head of a wearer.

2. The apparatus of claim 1 wherein said chamber is affixed to a cap.

3. The apparatus of claim 1 wherein said chamber is positioned adjacent to the crown of a cap.

4. The apparatus of claim 1 wherein said chamber includes a mechanism for closing and securing ice on the interior of the chamber

5. The apparatus of claim 1 wherein said chamber is at least partially porous to allow moisture from the interior of the chamber to diffuse into the porous member.

6. The apparatus of claim 4 wherein said diffusion of moisture occurs at least in part at the interface where said porous member is attached to said chamber.

7. The apparatus of claim 1 wherein said member has porosity to permit diffusion of water therethrough.
